(19) United States
(12) Patent Application Publication
(10) Pub. No.: US 2022/0218260 A1
(43) Pub. Date: Jul. 14, 2022

(54) SYSTEMS AND METHODS FOR QUANTITATIVE ECG HETEROGENEITY-GUIDED OPTIMIZATION OF THERAPEUTIC EFFICACY OF IMPLANTABLE CARDIAC DEVICES

(71) Applicant: Beth Israel Deaconess Medical Center, Inc., Boston, MA (US)

(72) Inventors: Richard L. VERRIER, Wellesley Hills, MA (US); Bruce D. Nearing, North Reading, MA (US); Peter J. ZIMETBAUM, Brookline, MA (US)

(73) Assignee: Beth Israel Deaconess Medical Center, Inc., Boston, MA (US)

(21) Appl. No.: 17/607,711

(22) PCT Filed: May 7, 2020
(86) PCT No.: PCT/US20/31864

§ 371 Date: Oct. 29, 2021

Related U.S. Application Data
(60) Provisional application No. 62/844,463, filed on May 7, 2019.

(51) Int. Cl. A61B 5/349; A61B 5/00; A61N 1/362

(52) U.S. Cl. CPC A61B 5/349; A61N 1/3627; A61B 5/7246

(57) ABSTRACT

Disclosed herein are example methods and systems for predicting efficacy of pacemakers or cardiac resynchronization therapy (CRT) devices prior to implantation in patients based on electrocardiogram (ECG) heterogeneity analysis. A method of determining or predicting efficacy of implanting a pacemaker or cardiac resynchronization therapy (CRT) device in a patient includes receiving a first set of electrocardiogram (ECG) signals associated with the patients heart from spatially separated leads, analyzing data from the first set of ECG signals, quantifying a spatio-temporal heterogeneity of the first set of ECG signals based on the analysis, and determining or predicting efficacy of implanting the pacemaker or cardiac re-synchronization therapy (CRT) device in the patient based on the quantified spatio-temporal heterogeneity.

### CROSS REFERENCE TO RELATED APPLICATIONS

This application claims the benefit under 35 U.S.C. § 119(e) of U.S. Provisional Application No. 62/844,463, filed May 7, 2019, which is hereby incorporated by reference in its entirety.

### BACKGROUND OF THE INVENTION

### Field of the Invention

Embodiments herein relate to systems and methods for predicting efficacy of pacemakers or cardiac resynchronization therapy (CRT) devices prior to implantation in patients based on electrocardiogram (ECG) heterogeneity analysis.

### Background

Cardiac resynchronization therapy (CRT) is an important treatment in patients with advanced heart failure and left ventricular dyssynchrony caused by left bundle branch block (LBBB). CRT has been proven to relieve symptoms, increase functional capacity, and prolong life in many cases, but the response rates ranged from 32% to 91% depending on the criteria used and were even lower if the strong placebo effect observed in the control group in most studies of CRT efficacy was subtracted from the treatment effect.

Despite availability of useful qualitative predictors of mechanical response to

CRT, including no prior history of myocardial infarction (MI) and non-ischemic etiology of cardiomyopathy, quantitative predictors, whether related to electrical or mechanical dyssynchrony, are suboptimal. Given the high cost and persistently high rate of nonresponse or suboptimal response to cardiac resynchronization therapy (CRT), more reliable quantitative predictors of response are needed. The most commonly used electrocardiographic criterion, QRS complex duration >150 ms, performs more poorly than qualitative clinical predictors. Therefore, more reliable quantitative predictors that might be useful independently or in combination with clinical indicators are needed. Additionally, there is a need for methods for determining optimum positioning of devices in a reliable and enhanced manner.

### SUMMARY OF THE INVENTION

Described herein are example methods and systems for quantitative ECG heterogeneity-guided optimization of therapeutic efficacy of implantable pacemaker and cardiac resynchronization therapy devices. Measurements and analysis of quantitative ECG heterogeneity may be utilized to improve the therapeutic efficacy of cardiac pacemakers and cardiac resynchronization therapy (CRT) devices in patients with heart failure. In particular, quantitative ECG heterogeneity may be utilized prior to implantation to determine which patients are likely to benefit from CRT. Quantitative ECG heterogeneity may also be utilized to guide placement of a lead in the coronary vein, as well as determining the optimum positioning of electrodes for improved stimulation efficacy.

In the embodiments presented herein, a method for determining or predicting efficacy of implanting a pacemaker or cardiac resynchronization therapy (CRT) device in a patient is described. The method includes receiving a first set of electrocardiogram (ECG) signals associated with the patient's heart from spatially separated leads, analyzing data from the first set of ECG signals, quantifying a spatio-temporal heterogeneity of the first set of ECG signals based on the analysis, and determining or predicting efficacy of implanting the pacemaker or cardiac resynchronization therapy (CRT) device in the patient based on the quantified spatio-temporal heterogeneity.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the present invention and to enable a person skilled in the relevant art(s) to make and use the present invention.

FIG. 1 illustrates an example diagram of leads of an ECG device placed on a patient, according to embodiments of the present disclosure.

FIG. 2 shows pre-implantation levels of R-wave and T-wave heterogeneity (RWH, TWH) as well as QRS complex duration in super-responders and non super-responders, according to embodiments of the present disclosure.

FIG. 3 provides representative patient tracings showing heterogeneity of R-wave and T-wave morphology (RWH, TWH) as well as QRS complex duration in one representative patient from the super-responder group and one representative patient from the non super-responder group, according to embodiments of the present disclosure.

FIG. 4 displays time course of R-wave and T-wave heterogeneity (RWH, TWH) and QRS complex duration at pre-implantation and at day one and at 3, 6, and 12 months after implantation, according to embodiments of the present disclosure.

FIGS. 5A, 5B, and 5C show receiver-operating characteristic (ROC) curves for the capacity of R-wave and T-wave heterogeneity (RWH, TWH) and QRS complex duration to predict mechanical super-response to cardiac resynchronization therapy (CRT) in the entire cohort (FIG. 5A) and in patients with (FIG. 5B) and without LBBB (FIG. 5C). RWH-_{V1-3} and TWH in all lead sets showed significance in the entire cohort, according to embodiments of the present disclosure.

FIGS. 6A and 6B provide Kaplan-Meier plots for all-cause mortality based on R-wave heterogeneity in the extended lead set (RWH_{V1-3LILII})≥420 V (FIG. 6A) and QRS complex duration >150 ms (FIG. 6B) in ECGs recorded prior to cardiac resynchronization therapy (CRT) device implantation in all 155 patients studied, according to embodiments of the present disclosure.

FIG. 7 shows an example ECG system configured to perform the electrocardiogram (ECG) heterogeneity procedures, according to embodiments of the present disclosure.

FIG. 8 shows a flowchart depicting a method for predicting efficacy of implanting a pacemaker or CRT device, according to embodiments of the present disclosure.

FIG. 9 shows an example computer system useful for implementing portions of the present disclosure.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

This specification discloses one or more embodiments that incorporate the features of this invention. The disclosed embodiment(s) merely exemplify the present invention. The scope of the present invention is not limited to the disclosed embodiment(s).

The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Embodiments of the present invention may be implemented in hardware, firmware, software, or any combination thereof. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

Before describing such embodiments in more detail, however, it is instructive to present an example environment in which embodiments of the present invention may be implemented.

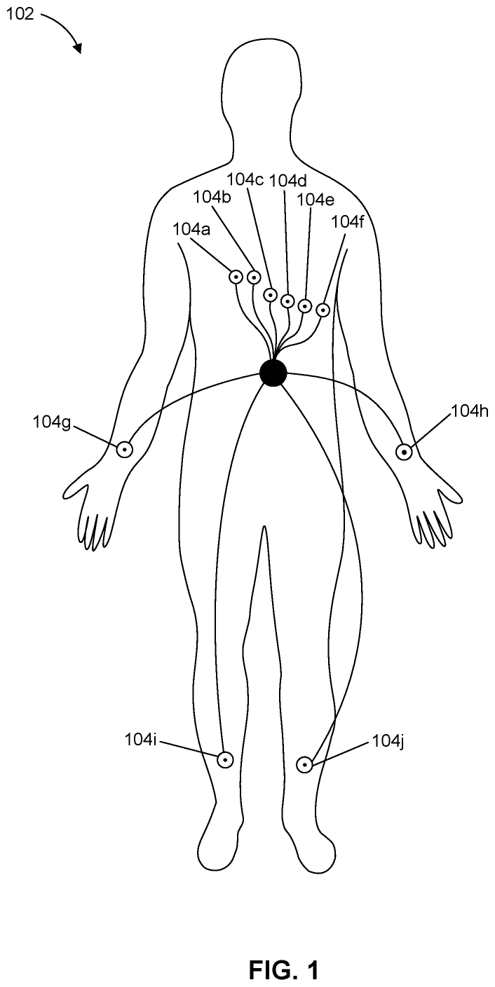

FIG. 1 illustrates a patient 102 that is attached to various leads of an ECG recording device, according to an embodiment. The leads may be used to monitor a standard 12-lead ECG. In this example, six leads (leads 104a-f) may be placed across the chest of patient 102 while four other leads (leads 104g-j) are placed with two near the wrists and two near the ankles of patient 102.

It should be understood that the exact placement of the leads is not intended to be limiting. For example, the two lower leads 104i and 104j may be placed higher on the body, such as on the outer thighs. In another example, leads 104g and 104h are placed closer to the shoulders while leads 104i and 104j are placed closer to the hips of patient 102. In still other examples, not all ten leads are required to be used in order to monitor ECG signals from patient 102.

In an embodiment, signals are monitored from each of leads 104a-j during a standard 12-lead ECG recording. The resulting ECG signal may be analyzed over time to determine various health factors such as heart rate, strength of heart beat, and any indicators of abnormalities. However, changes in the various signals received amongst leads 104a-j may be very small and difficult to detect. Any trend in the changing signal amplitude for certain areas of the ECG morphology could be vital in predicting a patient's response to cardiac resynchronization therapy (CRT). For example, prediction of efficacy of pacemakers or CRT devices prior to implantation in a patient may be possible by observing trends in the R-wave heterogeneity, T-wave heterogeneity, P-wave heterogeneity and/or T-wave alternans from the monitored ECG signals. In particular, ECG heterogeneity provides a spatial measure of T-wave wave morphology across different lead locations, and this metric may be at the root of dyssynchrony of mechanical contraction. Quantifying alternans in ECG signals is described in further detail in U.S. Pat. No. 6,169,919, which is incorporated by reference herein in its entirety.

The challenge is to separate these biologically significant microvolt-level changes from the intrinsic differences in ECG morphology. In an embodiment, the technique employed herein utilizes a multi-lead ECG median-beat baseline for each lead, which allows for the determination of ECG residua by subtraction of the baseline from the collected ECG signals. These residua may be evaluated in association with R-wave and T-wave heterogeneity analysis and other parameters for heart arrhythmia prediction, myocardial ischemia assessment, or determination of coronary artery stenosis.

Ultimately, the implementation of embodiments described herein may lead to improved identification of individuals/patients who are less likely or more likely to benefit from CRT and survive and may serve as a guide to determining efficacy of therapy.

Quantitative ECG heterogeneity may be used prior to implantation to determine which patients are likely to benefit from CRT over standard right ventricular (RV) pacing. Patients with a lower range of T-wave heterogeneity (TWH) respond more favorably to CRT implantation than those with high levels of TWH. Quantitative ECG heterogeneity may further be used to guide placement of a lead in the coronary vein of a patient as well as to determine the optimum electrode to be used in a quadripolar electrode catheter. In some embodiments, TWH may be used to determine optimum stimulation parameters.

ECG-guided CRT may also be helpful in determining optimum positioning of His bundle pacemaker electrodes compared to RV pacemaker electrodes. In particular, accurate positioning improves stimulation efficacy so that the activation wave will more uniformly propagate throughout the conducting system, and mechanical function will be synchronized.

In some embodiments, quantitative ECG heterogeneity-guided methods for optimization may be implemented for real-time monitoring in an electrophysiology study lab. For example, the electrophysiology study may include monitoring stations (e.g., computing devices, computer systems, or the like) which are coupled to ECG recording devices (e.g., configured with the leads shown in FIG. 1) to record full 12-lead ECGs of patients or individuals, as well as signals from cardiac catheters. In some embodiments, real-time monitoring of the quantified spatio-temporal heterogeneity may be visualized by an operator of the monitoring station on a user interface (e.g., a display, computer monitor, flat screen monitor, or the like).

In additional embodiments, methods for utilizing quantitative ECG heterogeneity to predict efficacy of pacemakers or CRT devices may be implemented in external stimulation units. For example, external stimulation units may be employed to optimize delivery of stimuli prior to implantation of a CRT or His bundle pacing device in a patient. These units may be equipped with software to provide a real-time readout of ECG heterogeneity. Once a predetermined or desired reduction in ECG heterogeneity is achieved using the external stimulation unit, the parameters and lead sites obtained from the heterogeneity readout data may be implemented in the implantable pacemaker or CRT device. In other words, the implantable pacemaker or CRT device may be implanted in a patient based on the parameters and lead sites identified during quantification of the ECG heterogeneity data.

### Study of ECG Heterogeneity for Predicting CRT Benefit and Survival

Cardiac resynchronization therapy (CRT) is an important treatment in patients with advanced heart failure and left ventricular dyssynchrony caused by left bundle branch block (LBBB). CRT has been proven to relieve symptoms, increase functional capacity, and prolong life in many cases, but the response rates ranged from 32% to 91% depending on the criteria used and were even lower if the strong placebo effect observed in the control group in most studies of CRT efficacy was subtracted from the treatment effect.

Despite availability of useful qualitative predictors of mechanical response to

CRT, including no history of myocardial infarction or non-ischemic etiology of cardiomyopathy, quantitative predictors, whether related to electrical or mechanical dyssynchrony, may be suboptimal. The most commonly used electrocardiographic (ECG) criterion, QRS complex duration, may perform more poorly than qualitative clinical predictors. Therefore, more reliable quantitative predictors that might be useful independently or in combination with clinical indicators are desirable.

The present retrospective study tested the hypothesis that quantitative assessment of R-wave and T-wave heterogeneity (RWH and TWH, respectively) at pre-implantation could be employed both in predicting mechanical super-response to CRT and in assessing mortality risk. The scientific rationale underlying prediction of mechanical response is based on the close linkage between nonuniformities in excitation and contraction coupling in diseased myocardium and the recent finding that mechanical dyssynchrony as assessed by global longitudinal strain is significantly correlated with TWH. The inference for mortality risk prediction in this population derives from two recent studies. The first was the sizeable Health Survey 2000 study of 5600 subjects in which TWH predicted adjudicated SCD as well as total and cardiovascular mortality (Kenttä T V, et al 2016). The second study enrolled patients with cardiomyopathy from the institution in whom RWH and TWH in 12-lead ECG recordings were found to predict sustained ventricular arrhythmia, appropriate implantable cardioverter defibrillator (ICD) therapies, and arrhythmic death or cardiac arrest independent of age, sex, and left ventricular ejection fraction (LVEF) (Tan A Y, et al. 2017).

The present study involved direct comparison of RWH and TWH against the mainstay clinical ECG variable, QRS complex duration, for prediction of both mechanical super-response to CRT and survival.

### Methods

### Study Population

The inclusion criteria for this retrospective study were Class I and IIA ACC/AHA/HRS guideline-based indications for CRT device implantation (LBBB, QRS complex duration ≥120 ms, LVEF≥35% and New York Heart Association (NYHA) functional class≥II or non-LBBB, QRS≥150 ms, LVEF≤35%, NYHA functional class ≥III); echocardiograms before (average 4.1±1 months) and at one year after implantation; and ECGs before implantation and at least one of four post-implantation time points (1 day, 3 months, 6 months, and 1 year). Of the patients who received CRT devices from 2006 to 2018 at the institution, ECGs from all 155 patients who met these criteria were retrospectively analyzed. This investigation was approved as a medical records study by the Institutional Review Board of Beth Israel Deaconess Medical Center.

Left ventricular systolic dysfunction was categorized as being due to coronary artery disease (CAD) if there was regional wall motion abnormality and/or scar on echocardiography, cardiac magnetic resonance imaging, or nuclear stress imaging indicative of prior myocardial infarction, and any of the following criteria were fulfilled: (1) documented prior myocardial infarction, (2) >70% stenosis of a major epicardial coronary artery, (3) documented prior coronary artery bypass graft surgery, or (4) documented prior percutaneous coronary intervention. All other patients with left ventricular systolic dysfunction were considered to have a nonischemic etiology.

Super-responders (n=35) were defined as having a ≤20% increase in LVEF and/or ≥20% decrease in left ventricular end-systolic diameter (LVESD) across 1 year while non super-responders (n=120) did not meet these criteria. These criteria were based on the echocardiographic results obtained by Rickard et al (2010) in their investigation of potential predictors of super-response. See Rickard J, Kumbhani D J, Popovic Z, et al. Characterization of super-response to cardiac resynchronization Therapy. Heart Rhythm 2010;7:885-889. A subset analysis using a 5% LVEF cut off was also performed.

### ECG and Echocardiographic Analyses

Interlead heterogeneity of depolarization and repolarization morphology was assessed from 10-sec 12-lead ECG recordings (GE Healthcare, Milwaukee Wis., USA) by an investigator blinded to clinical status and outcomes using second central moment analysis. This method quantifies variability about the mean morphology of ECGs from adjoining leads on a beat-to-beat basis. Specifically, after the signals are processed to filter noise and remove baseline wander, the software generates mean waveforms separately for the QRS complex and T waves (to include the J point and entire T wave) of adjoining precordial leads V₁₋₃ (RWH_{V1-3}, TWH_{V1-3}) and V₄₋₆ (RWHV₄₋₆, TWH_{V4-6}) and also including leads I and II (RWH_{V1-3LILII}, RWH_{V4-6LILII}, TWH_{V1-3LILII}, TWH_{V4-6LILII}). Use of the extended lead sets enhances the electrophysiologic field of view for ECG heterogeneity determination. This mean interlead morphology constitutes the first moment or central axis in the terminology of Newtonian physics. The second central moment, or mean-square deviation, was then determined to quantify the variability or splay about the mean morphology. Finally, the maximum square root of the second central moment was calculated to obtain the RWH and TWH values in microvolts. Using this analytic technique, ECG heterogeneity measurement is not unduly weighted by protracted termination or inflections in the waveforms, ST-segment changes, or presence of U waves, features that limit accurate dispersion measurement by conventional analyses. The maximum RWH and TWH levels for each patient were determined.

QRS complex duration was determined in the longest representative non-premature beat and the clinical criterion of ≥150 ms was used. All ECG parameters were analyzed by investigators blinded to the echocardiographic parameters and clinical characteristics.

LVEF was assessed by transthoracic echocardiography (GE Healthcare, Hartford, Conn., USA) and measured by biplane Simpson's method.

### Statistics

Statistical analysis was performed with XLSTAT (Addinsoft, Inc., New York, N.Y., USA) and GraphPad Prism (San Diego, Calif., USA). Data are reported as means±standard error of the mean. Statistical differences in quantitative measurements between super-responders and non super-responders were calculated using 2-tailed unpaired Student's t-test or Welch's t-test, chosen according to differences in variances calculated by F-test as well as by Mann-Whitney's test for non-parametric data. Differences in qualitative measurements were calculated using Fisher's exact test. Receiver-operating characteristic (ROC) curves were plotted for the ECG parameters, and the cutpoints for RWH, TWH, and QRS complex duration were chosen as the most optimized value, that is, the measurement level with the maximum sensitivity plus specificity. Logistic regression was used to calculate odds ratios, with multivariate analysis being used to adjust the ECG parameters for confounding by clinical parameters in which the difference between the groups had a p-value of <0.1. Two-way repeated measures analysis of variance (ANOVA) was used to analyze progression of ECG parameters over time in the two groups of patients. Kaplan-Meier curves for mortality were determined and statistically evaluated using the log-rank test.

### RESULTS

### Clinical Characteristics

The clinical characteristics of super-responders (N=35) and non super-responders (N=120) are presented in Table 1.

TABLE 1
Baseline patient characteristics

| | Super- | | Non Super- | | |
|---|---|---|---|---|---|
| | Responders | | Responders | | |
| | (n = 35) | | (n = 120) | | p value |
| Sex (M/F) | 22/13 | | 91/29 | | 0.13 |
| Age (mean, SEM) | 66.2 ± 2 | | 69.8 ± 1 | | 0.13 |
| Device Type (CRT-P/CRT-D) | 11/24 | | 31/89 | | 0.52 |
| Programming (DDD/VVI) | 30/5 | | 106/14 | | 0.76 |
| Device upgrade (n, %) | 11 | (31) | 55 | (46) | 0.17 |
| CAD (n, %) | 14 | (40) | 77 | (64) | 0.02 |
| MI Hx (n, %) | 7 | (20) | 48 | (40) | 0.04 |
| HTN (n, %) | 21 | (60) | 92 | (77) | 0.08 |
| AF (n, %) | 16 | (46) | 55 | (46) | 1 |
| DM II (n, %) | 11 | (31) | 53 | (44) | 0.24 |
| LBBB/Non-LBBB | 29/6 | | 91/29 | | 0.49 |
| ICM/NICM | 10/25 | | 57/53 | | 0.02 |
| NYHA (mean ± SEM) | 2.7 ± 0.1 | | 3.0 ± 0.2 | | 0.24 |
| NYHA after 1 year (mean ± SEM) | 1.5 ± 0.1 | | 1.9 ± 0.1 | | 0.01 |
| Δ NYHA at 1 year (mean ± SEM) | −1.2 ± 0.1 | | −1.1 ± 0.3 | | 0.74 |
| Pacing % at 1 year (mean ± SEM) | 97.4 ± 0.7 | | 95.8 ± 0.7 | | 0.12 |
| Diuretics (n, %) | 28 | (80) | 106 | (89) | 0.26 |
| Beta-blockers (n, %) | 27 | (77) | 108 | (90) | 0.09 |
| ARB (n, %) | 12 | (34) | 27 | (23) | 0.19 |
| ACE inhibitors (n, %) | 16 | (46) | 63 | (53) | 0.56 |
| Aldosterone inhibitors (n, %) | 10 | (29) | 37 | (31) | 0.83 |
| Calcium channel blockers (n, %) | 3 | (9) | 9 | (8) | 0.73 |
| Amiodarone (n, %) | 5 | (14) | 25 | (21) | 0.47 |
| Statins (n, %) | 23 | (66) | 84 | (74) | 0.68 |
| Digitalis (n, %) | 8 | (23) | 21 | (18) | 0.47 |

Abbreviations: ACE = angiotensin converting enzyme; AF = Atrial Fibrillation; ARB = Angiotensin II Receptor Blocker; CAD = Coronary Artery Disease; CRT-D: Cardiac Resynchronization Therapy-Defibrillator; CRT-P: Cardian Resynchronization Therapy-Pacemaker; DM II = Type II Diabetes; F = female; HTN = Hypertension; ICM = Ischemic Cardiomyopathy; LBBB = left bundle branch block; M = male; MI Hx = History of Myocarial Infarction; NICM = Non-Ischemic Cardiomyopathy; NYHA = New York Heart Associate functional class; SEM = standard error of the mean.

Non super-responders exhibited a significantly higher prevalence of CAD, history of myocardial infarction, and ischemic cardiomyopathy, which are well-established predictors of poor response to CRT. The p value for the difference in hypertension and use of beta blockers was <0.1; therefore, these criteria were included in the multi-variate analysis as well. Significant differences in characteristics after 1 year comparing super-responders to non super-responders included NYHA functional class (1.5±0.1 vs 1.9±0.1, respectively, p=0.01). The number of patients with 12-lead ECG recordings at each time point are presented in Table 2.

TABLE 2
Number of patients with ECGs and each time point

| | Pre-CRT | Day | 3 | 6 | 1 |
|---|---|---|---|---|---|
| | implantation | after | months | months | year |
| Super-Responders | 35 | 24 | 19 | 19 | 20 |
| Non Super-Responders | 120 | 83 | 71 | 65 | 67 |

### Echocardiogram Parameters

There were no significant differences in the pre-implantation echocardiographic parameters when comparing the super-responders and non super-responders (Table 3). In post-treatment measurements, there were significant differences between super-responders and non super-responders in terms of LVEF, LVESD, left ventricular end-diastolic diameter (LVEDD), and mitral valve regurgitation.

TABLE 3
Changes in echocardiographic characteristics across time

| | Non Super-Responders | | | Super-Responders | | | p value | | |
|---|---|---|---|---|---|---|---|---|---|
| | Pre-CRT | Post-CRT | | Pre-CRT | Post-CRT | | Pre-CRT | Post-CRT | |
| | device | device | | device | device | | device | device | |
| | implantation | implantation | Change | implantation | implantation | Change | implantation | implantation | Change |
| LVEF, % (n) | 24.2 ± 0.6 | 28.2 ± 0.8 | 4 ± 0.6 | 23 ± 1.1 | 50.2 ± 1.3 | 27.2 ± 1.5 | 0.32 | <0.0001 | <0.0001 |
| | (120) | (120) | (120) | (35) | (35) | (35) | | | |
| LVESD, mm (n) | 52.6 ± 1.6 | 52.1 ± 1.3 | −0.8 ± 1 | 51.8 ± 2.4 | 33.1 ± 1.6 | −15.6 ± 1.5 | 0.74 | <0.0001 | <0.0001 |
| | (71) | (62) | (38) | (18) | (16) | (8) | | | |
| LVEDD, mm (n) | 61.5 ± 0.8 | 59.1 ± 0.9 | −2.4 ± 0.6 | 59.2 ± 1.6 | 49.1 ± 1.3 | −9.9 ± 1.5 | 0.17 | <0.0001 | <0.0001 |
| | (116) | (114) | (111) | (34) | (35) | (34) | | | |
| MVR, +/4+ (n) | 1.5 ± 0.1 | 1.3 ± 0.1 | −0.2 ± 0.1 | 1.4 ± 0.1 | 0.9 ± 0.1 | −0.5 ± 0.2 | 0.47 | 0.01 | 0.13 |
| | (119) | (118) | (117) | (34) | (34) | (33) | | | |
| LAD, mm (n) | 60.9 ± 0.8 | 59.4 ± 1 | −1.2 ± 1.1 | 58.7 ± 1.7 | 56.7 ± 1.8 | −1.1 ± 2.0 | 0.19 | 0.19 | 0.97 |
| | (110) | (107) | (100) | (30) | (32) | (27) | | | |

Abbreviations:
CRT = cardiac resynchronization therapy;
LAD = Left Atrium 4-Chamber Diameter;
LVEDD = Left Ventricular End-Diastolic Diameter;
LVEF = Left Ventricular Ejection Fraction;
LVESD = Left Ventricular End-Systolic Diameter;
MVR = Mitral Valve Regurgitation

### ECG Parameters

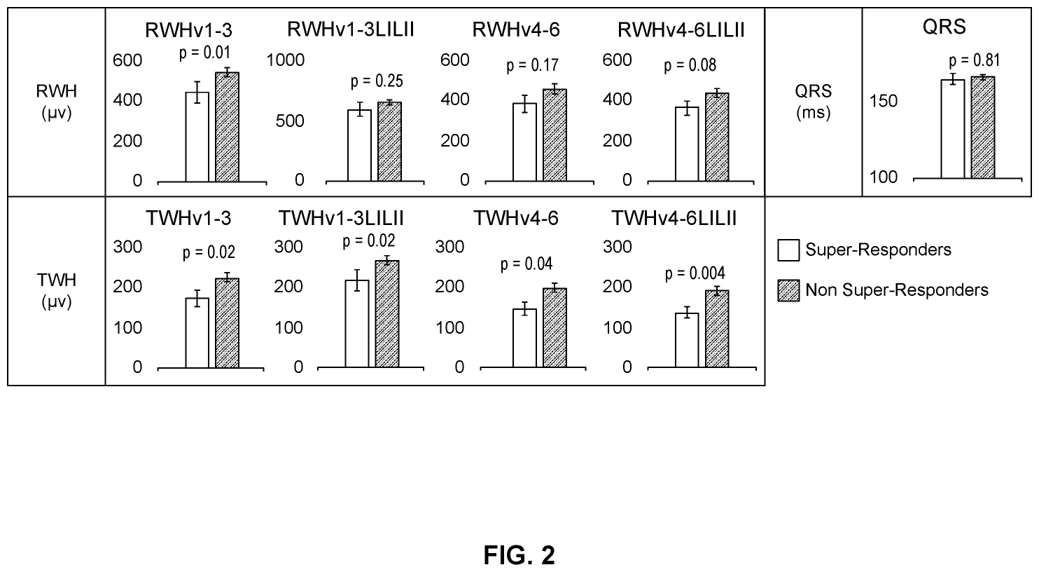

FIG. 2 provides pre-implantation levels of R-wave and T-wave heterogeneity

(RWH, TWH) as well as QRS complex duration in super-responders and non super-responders. Pre-implantation RWH_{V1-3} and all TWH lead combinations were significantly lower in super-responders compared to non super-responders [(RWH_{V1-3} (442±51 vs 542±23 μV; p=0.01), TWH_{V1-3} (168±19 vs 219±11 μV; p=0.02), TWH_{V1-3LILII} (80±9 vs 217±23 μV; p=0.02), TWH_{V4-6} (146±16 vs 198±11 μV; p=0.04), and TWH_{V4-6LILII}(136±12 vs 190±9 μV; p=0.004)]. Pre- implantation RWH_{V1-3LILII} (595±54 vs 651±23 μV; p=0.26), RWH_{V4-6} (383±44 vs 458±26 μV; p=0.17), and RWH_{V4-6LILII} (363±34 vs 438±21 μV; p=0.08) did not differ between super-responders and non-super-responders.

Pre-implantation QRS complex duration did not differ between super-responders and non super-responders (166±3 vs. 167±2 ms, p=0.81). Lower pre-implantation RWH_{V1-3} (470±23 vs 563±37 p=0.04) and TWH_{V1-3} levels (185±10 vs 241±20 p=0.009) were also associated with positive response to CRT using the less restrictive 5% LVEF increase criterion.

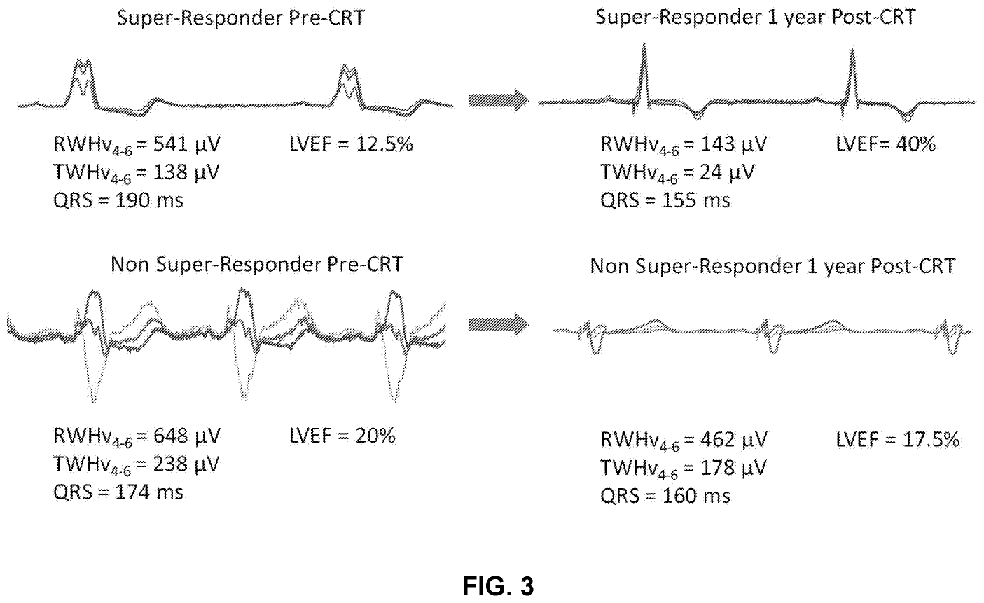

FIG. 3 shows superimposed ECGs from leads V₄₋₆ from one representative super-responder and one representative non super-responder demonstrating reductions in RWH, TWH, and QRS complex duration comparing baseline to 1 year after implantation.

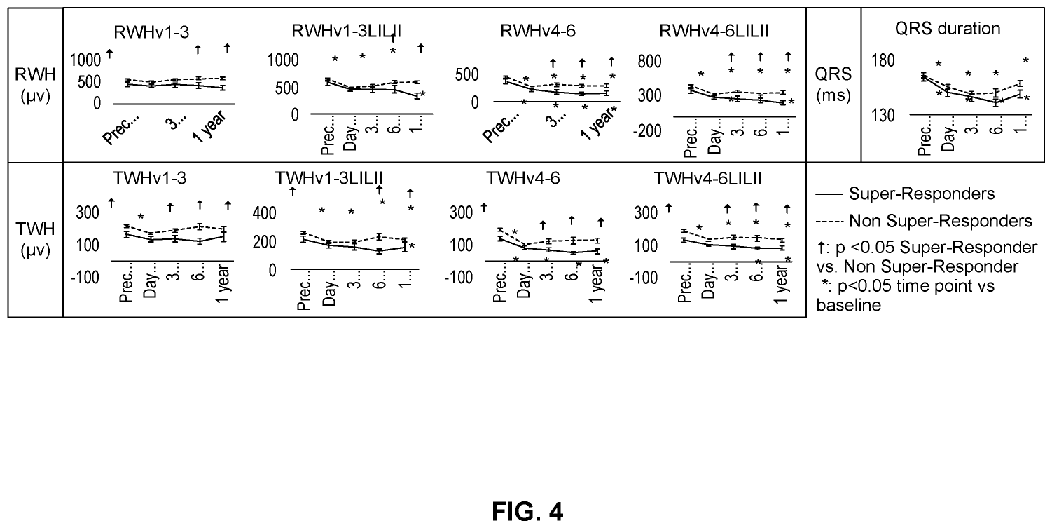

In the entire cohort, at follow-up after one year, RWH and TWH in all lead sets tested were significantly lower among super-responders than non super-responders (FIG. 4), but there was no significant difference between the groups in QRS complex duration (150±4 vs 159±3 ms, p=0.28). Specifically, FIG. 4 shows the time course of R-wave and T-wave heterogeneity (RWH, TWH) and QRS complex duration at pre-implantation and at day one and at 3, 6, and 12 months after implantation, according to embodiments of the present disclosure. Numerical data for significant differences in lead sets at 1 year following cardiac resynchronization therapy (CRT) device implantation are: RWH_{V1-3} (349±56 vs 577±33 p=0.005), RWH_{V1-3LILII} (340±52 vs 593±29 p=0.0001), RWH_{V4-6} (163±30 vs 300±36 μV, p=0.007), RWH_{V4-6LILII} (196±27 vs 344±27 μV, p=0.0008), TWH_{V1-3} (153±29 vs 202±14 μV, p=0.01), TWH_{V1-3LILII} (159±33 vs 213±13 μV, p=0.003), TWH_{V4-6} (67±15 vs 133±16 μV, p=0.004), and TWH_{V4-6LILII} (84±12 vs 137±13 μV, p=0.003). †p<0.05 comparing super-responders and non super-responders. *p<0.05 comparing the time point and baseline.

### Prediction of CRT Super-Response

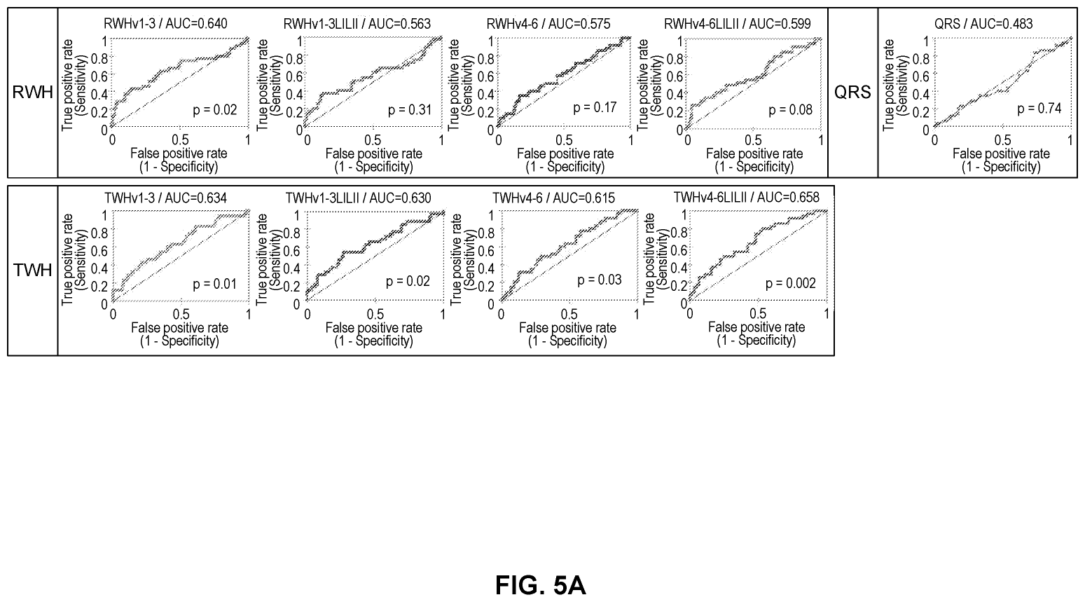

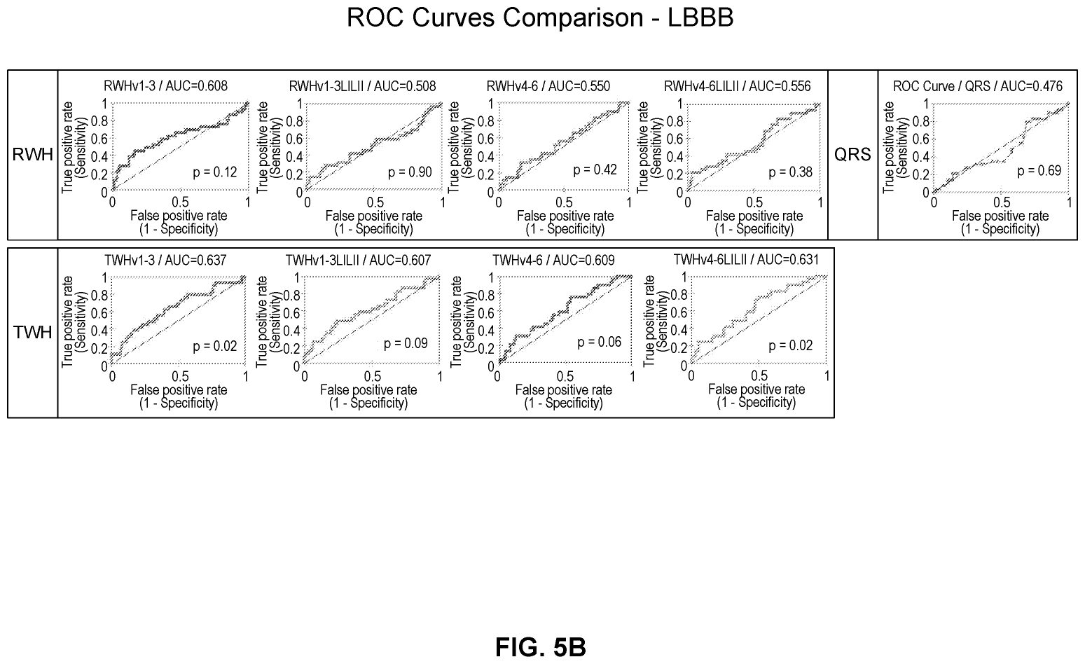

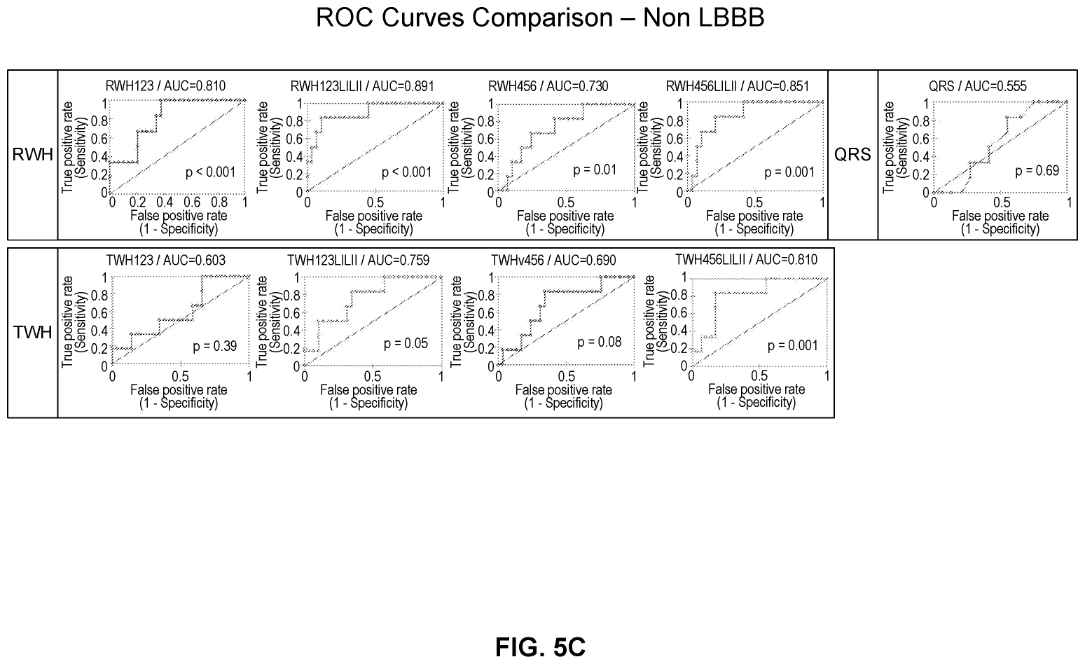

FIGS. 5A, 5B, and 5C illustrate example diagrams showing receiver-operating characteristic (ROC) curves for the capacity of R-wave and T-wave heterogeneity (RWH, TWH) and QRS complex duration to predict mechanical super-response to cardiac resynchronization therapy (CRT) in the entire cohort (FIG. 5A) and in patients with (FIG. 5B) and without LBBB (FIG. 5C). RWH_{V1-3} and TWH in all lead sets showed significance in the entire cohort, according to embodiments of the present disclosure. The p-value is based on difference between the areas under the curve (AUC) in each graph and a 0.5 (random) AUC.

Specifically, areas under the curve (AUC) were significantly different from random in RWH_{V1-3} (p=0.02) and in all TWH lead sets tested (p=0.002 to 0.03) (FIG. 5A). However, the AUC for QRS complex duration (0.48) did not differ significantly from random (p=0.74).

Table 4 shows the unadjusted odds ratios, optimized cutpoints, and test performances for ECG parameters in predicting super-response.

TABLE 4
Performance of different pre-implantation criteria in predicting mechanical
super-response to cardiac resynchronization therapy (Unadjusted Odds Ratios)

| Criteria | Cutpoint | OR | CI 95% | P | Sensitivity | Specificity | PPV | NPV |
|---|---|---|---|---|---|---|---|---|
| RWHᵥ₁₋₃ (μV) | 296 | 4.54 | 1.95-10.56 | 0.0004 | 42.9 | 85.8 | 46.9 | 83.7 |
| RWH_{v1-3LILII} (μV) | 408 | 3.84 | 1.61-9.11 | 0.002 | 37.1 | 86.7 | 44.8 | 82.5 |
| RWHᵥ₄₋₆ (μV) | 198 | 2.29 | 0.97-5.42 | 0.06 | 34.3 | 83.3 | 37.5 | 81.3 |
| RWH_{v4-6LILII} (μV) | 164 | 6.58 | 2.15-20.1 | 0.001 | 25.7 | 95 | 60 | 81.4 |
| TWHᵥ₁₋₃ (μV) | 119 | 2.85 | 1.28-6.35 | 0.01 | 42.9 | 79.2 | 37.5 | 82.6 |
| TWH_{v1-3LILII} (μV) | 182 | 3.27 | 1.5-7.11 | 0.003 | 54.3 | 73.3 | 37.3 | 84.6 |
| TWHᵥ₄₋₆ (μV) | 116 | 2.29 | 1.06-4.96 | 0.03 | 48.6 | 70.8 | 32.69 | 82.5 |
| TWH_{v4-6LILII} (μV) | 176 | 3.5 | 1.42-8.63 | 0.006 | 80 | 46.7 | 30.4 | 88.9 |
| QRS (ms) | 150 | 1.2 | 0.45-3.24 | 0.71 | 85.7 | 16.7 | 23.1 | 80 |
| QRS (ms) | 155 | 1.75 | 0.67-4.62 | 0.25 | 82.9 | 26.7 | 24.8 | 84.2 |
| NO CAD | | 2.69 | 1.25-5.81 | 0.01 | 60 | 64.2 | 32.8 | 84.6 |
| NO MI Hx | | 2.67 | 1.08-6.6 | 0.04 | 80 | 40 | 28 | 87.3 |
| NO HTN | | 2.19 | 0.98-4.86 | 0.05 | 40 | 76.7 | 33.3 | 81.4 |
| NICM | | 3.16 | 1.39-7.15 | 0.005 | 71.4 | 55.8 | 32 | 87 |
| No Beta-blockers | | 2.67 | 0.99-7.17 | 0.05 | 22.9 | 90 | 40 | 80 |

Abbreviations: OR = odds ratio; PPV = positive predictive value; NPV = negative predictive value; CAD = coronary artery disease; HTN = hypertension; MI Hx = history of myocardial infarction; NICM = non-ischemic cardiomyopathy.
Bold font indicates significant odds ratios.

When adjusted for confounding by absence of CAD, hypertension, and history of acute myocardial infarction, and presence of nonischemic cardiomyopathy (Table 5), the optimized cutpoints for RWH and TWH in all lead sets yielded significant odds ratios (2.91 to 13.05, p<0.0001 to 0.02) for predicting mechanical super-response to CRT. RWH_{V4-6LILII} produced the highest odds ratio and greatest significance level. Neither the standard (150 ms) nor the optimized cutpoint (155 ms) for QRS complex duration yielded a significant odds ratio.

TABLE 5
Adjusted odds ratios for performance of different pre-implantation
criteria in predicting mechanical super-response to CRT
Adjusted Odds Ratios*

| Criteria | cutpoint | OR | CI 95% | P |
|---|---|---|---|---|
| RWHᵥ₁₋₃ (μV) | 296 | 7.82 | 2.86-21.36 | 0.0001 |
| RWH_{v1-3LILII} (μV) | 408 | 6.21 | 2.29-16.86 | 0.0003 |
| RWHᵥ₄₋₆ (μV) | 198 | 2.91 | 1.14-7.44 | 0.02 |
| RWHV_{4-6LILII} (μV) | 164 | 13.05 | 3.58-47.56 | <0.0001 |
| TWHᵥ₁₋₃ (μV) | 119 | 4.02 | 1.62-9.93 | 0.003 |
| TWH_{v1-3LILII} (μV) | 182 | 4.2 | 1.77-9.94 | 0.001 |
| TWHᵥ₄₋₆ (μV) | 116 | 3.03 | 1.3-7.08 | 0.01 |
| TWH_{v4-6LILII} (μV) | 176 | 5.38 | 1.98-14.64 | 0.001 |
| QRS complex (ms) | 150 | 1.66 | 0.58-4.74 | 0.35 |
| QRS complex (ms) | 155 | 2.4 | 0.86-6.7 | 0.09 |

Odds ratios were adjusted for absence of coronary artery disease, history of acute myocardial infarction, and hypertension, and presence of non-ischemic cardiomyopathy. RWH=R-wave heterogeneity; TWH-T-wave heterogeneity.

### Prediction of All-Cause Mortality

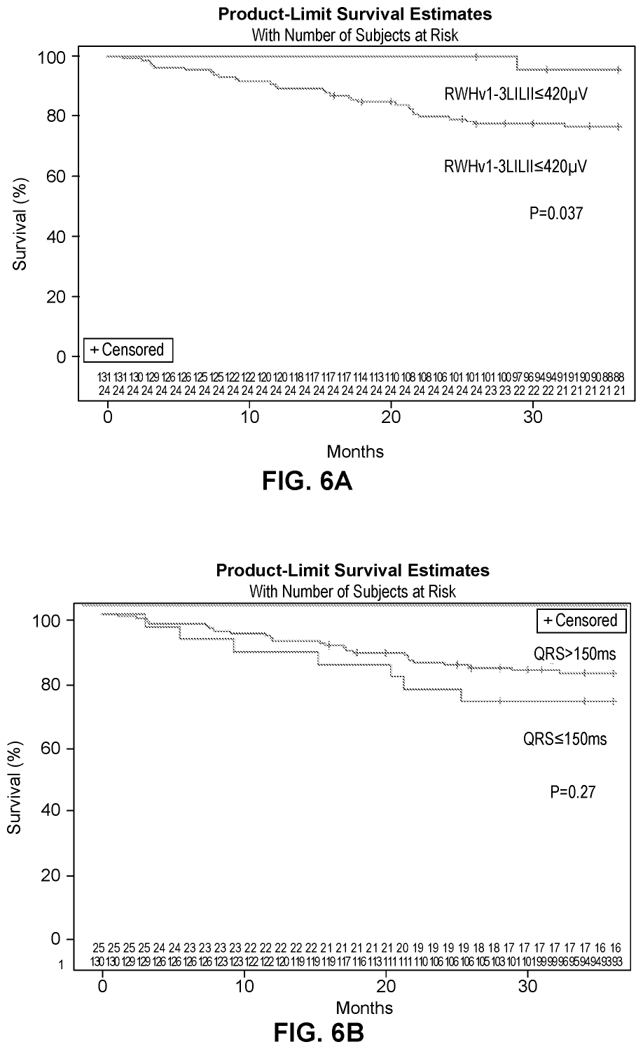

During the follow-up period of 3 years, there were 39 deaths (32.5%) among the 120 non super-responders and 7 deaths (20%) among the 35 super-responders (p=0.005). Kaplan-Meier analysis revealed that 3-year mortality was significantly increased in patients with pre-implantation RWH_{V1-3LILII}≥420 μV (p=0.037) with a hazard ratio of 7.440 (95% CI: 1.015-54.527, p=0.048) (shown in FIG. 6A) in ECGs recorded prior to cardiac resynchronization therapy (CRT) device implantation, according to embodiments of the present disclosure. In this analysis, neither pre-implantation QRS complex duration>150 ms (p=0.27) (shown in FIG. 6B) nor TWH in any lead set predicted 3-year all-cause mortality (p=0.20).

### Discussion

Main findings

The study demonstrated that reduced levels of ECG repolarization heterogeneity at pre-implantation predict mechanical response to CRT and survival. The findings indicate that RWH_{V1-3} and TWH in all lead combinations are superior to QRS complex duration, the mainstay ECG marker currently used to predict response to CRT. This predictive capacity remains significant even after adjustment for clinical characteristics. Furthermore, the results reveal that CRT reduced RWH and TWH in all patients, particularly among super-responders. Importantly, RWH_{V1-3LILII}≥420 μV was associated with increased risk of total 3-year mortality (p=0.037) with a hazard ratio of 7.440 (95% CI: 1.015-54.527, p=0.048).

### Prior Studies

Preclinical studies reported an increase in QT interval and transmural dispersion of repolarization (Tₚₑₐₖ-T_{end} interval) in ventricular wedge preparations during epicardial pacing. Fish, Di Diego, Nesterenko, and Antzelevitch (2004) made the fundamental observation that under conditions in which QT interval is prolonged, epicardial to endocardial activation may predispose to malignant arrhythmias such as torsades de pointes by increasing transmural dispersion of repolarization. See Fish J M, Di Diego J M, Nesterenko V, Antzelevitch C. Epicardial activation of left ventricular wall prolongs QT interval and transmural dispersion of repolarization. Implications for biventricular pacing. Circulation 2004;109:2136-2142. Clinical studies have indicated a decrease in heterogeneity in terms of QT dispersion and/or Tpeak-Tend interval, etc., following CRT, especially in the long-term. Cvijic and colleagues (2018) reported in a prospective study of 64 patients that lower repolarization heterogeneity in terms of QT interval, Tpeak-Tend, and Tpeak-Tend/QT ratio characterized patients with better mechanical response to CRT at one year following implantation. See Cvijic M, Antolic B, Klemen L, Zupan I. Repolarization heterogeneity in patients with cardiac resynchronization therapy and its relation to ventricular tachyarrhythmias. Heart Rhythm 2018;15:1784-1790. Moreover, reduced Tpeak-Tend/QT ratio distinguished patients with lower incidence of ventricular tachycardia and ventricular fibrillation during follow-up. However, they did not report that any of these indices at pre-implantation helped to distinguish patients with improved mechanical response to CRT or improved survival.

### Current Study

The central question addressed is whether or not RWH and/or TWH is superior to the mainstay measurement of QRS complex duration in predicting mechanical super-response and mortality in patients receiving CRT devices. The rationale for the study was the well-established excitation-contraction coupling relationship as well as a recent finding in patients with newly developed conduction abnormalities after transcatheter aortic valve replacement that TWH is significantly correlated with mechanical dyssynchrony as measured by global longitudinal strain. Thus, it was hypothesized that TWH could help to reveal electro-mechanical dyssynchrony and global cardiac pathology and therefore to predict which patients might not experience enhanced mechanical response to CRT. Also, in light of studies showing the capacity of TWH to predict sudden cardiac death and cardiac and total mortality, it was postulated that RWH and/or TWH at the time of CRT implantation would predict premature demise.

Pre-implantation levels of RWH and TWH in some leads were found to be superior to QRS complex duration in predicting mechanical super-response to CRT (Table 5). The optimized RWH and TWH cutpoints yielded significant odds ratios that were >7-fold greater than the non-significant odds ratio produced by QRS complex duration of 150 ms (Table 5). Furthermore, at the end of 1-year follow up, the super-responders were found to have significantly lower levels of RWH and TWH in all analyzed lead sets than the non super-responders, while QRS complex duration did not discriminate between the groups (FIG. 4). Moreover, the AUC for prediction of super-response by RWH and TWH was significant while the AUC achieved by QRS complex duration was not (FIGS. 5A, 5B, and 5C). It is noteworthy that low RWH and TWH levels also predicted CRT response in both the presence and absence of LBBB (FIGS. 5B and 5C). These observations carry important practical implications in light of evidence that patients with non-LBBB may benefit from CRT (19). The ENHANCE-CRT trial is designed to shed further light on this issue (Singh et al 2018). See Singh J P, Berger R D, Doshi R N, Lloyd M, Moore D, Daoud E G, for the ENHANCE CRT study group. Rationale and design for ENHANCE CRT: QLV implant strategy for non-left bundle branch block patients. ESC Heart Failure 2018;4:1184-1190.

A major finding of the present study is that quantification of RWH in leads V1-3, LI, and LII, from a single 12-lead ECG recorded at the pre-implantation of a CRT device predicted overall mortality during a 36-month follow-up (FIGS. 6A and 6B). By comparison, the Kaplan-Meier curve for QRS complex duration ≥150 ms did not achieve statistical significance nor did TWH in in any of the lead sets. The basis for the superiority of RWH in predicting survival is unclear. A possible explanation is that depolarization exerts a greater influence on mechanical synchrony than does repolarization. It is unclear why lower levels of RWH are associated with super-response. A potential explanation is that excessive degrees of heterogeneity may inhibit the capacity to confer improvement in electrical and mechanical dyssynchrony. Detailed study of the excitation-contraction relationships in patients with implanted CRT devices will be required to address this question and the underlying mechanisms.

The capacity to obtain a significant level of prediction from a single, routine 12-lead ECG recording is consistent with prior studies. Specifically, in Health Survey 2000, ECG heterogeneity achieved odds ratios of 3.2 among the 5600 individuals surveyed as representative of the entire Finnish population who underwent a routine health screening (Kentta et al 2016). See Kenttä T V, Nearing B D, Porthan K, Tikkanen J T, Viitasalo M, Nieminen M S, Salomaa V, Oikarinen L, Huikuri H V, Verrier R L. Prediction of sudden cardiac death with automated high throughput analysis of heterogeneity in standard resting 12-lead electrocardiogram. Heart Rhythm 2016; 13:713-720. Tan et al (2017) found that RWH and/or TWH recorded from a single 12-lead ECG obtained in the cardiac electrophysiology study laboratory at the time of ICD implantation or battery replacement predicted arrhythmia-free and total survival independent of age, sex, and LVEF. See Tan A Y, Nearing B D, Rosenberg M, Nezafat R, Josephson M E, Verrier R L. Interlead heterogeneity of R- and T-wave morphology in standard 12-lead ECGs predicts sustained ventricular tachycardia/fibrillation and arrhythmic death in patients with cardiomyopathy. J Cardiovasc Electrophysiol 2017; 28:1324-1333.

An important question for future investigation is the precise electrophysiologic basis for the capacity of pre-implantation RWH and TWH to predict super-mechanical response, especially in comparison to QRS complex duration. A salient feature of RWH and TWH is that they provide temporo-spatial information as they involve signals from 3 to 5 leads compared to a single lead in the case of QRS complex duration. There is mounting evidence that interlead heterogeneity of R- and T-wave morphology is more informative than conventional markers of repolarization abnormality, such as QT prolongation and dispersion, which are affected by the uncertain determination of the end of the T wave (Kentta et al 2016; Verrier and Huikuri 2017; Porthan et al 2013). See Verrier R L, Huikuri H V. Tracking interlead heterogeneity of R- and T-wave morphology to disclose latent risk for sudden cardiac death. Heart Rhythm 2017; 14:1466-1475; Porthan K, Viitasalo M, Toivonen L, et al. Predictive value of electrocardiographic T-wave morphology parameters and T-wave peak to T-wave end interval for sudden cardiac death in the general population. Circ Arrhythm Electrophysiol 2013;6:690-696. The basic assumption is that multilead ECG morphology analysis provides an improved assessment of the underlying action potential patterns and their linkage to contractile synergy through the biophysical factors at the root of excitation-contraction coupling. In other words, the greater the electrical dyssynchrony, the greater the mechanical dyssynchrony. The underlying principle of CRT is to mitigate this adverse condition.

### CONCLUSION

Pre-implantation ECG heterogeneity is superior to QRS complex duration in predicting mechanical super-response to CRT and survival. Patients with higher RWH and TWH levels are less likely to benefit from CRT than those with lower levels. Ultimately, ECG heterogeneity could also be utilized, along with clinical and echocardiographic parameters, to monitor patients' response to treatment.

### Exemplary Embodiments of ECG Systems and Methods of Operation

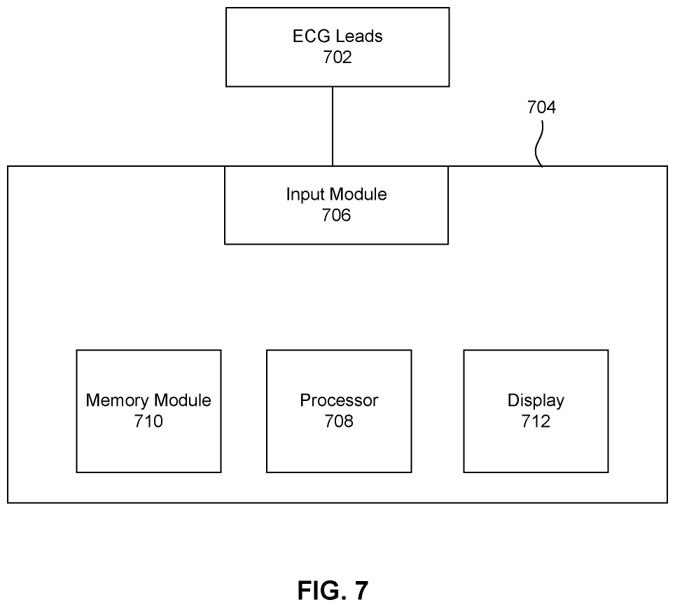

FIG. 7 illustrates an example ECG system 700 configured to perform the electrocardiogram (ECG) heterogeneity procedures, according to embodiments of the present disclosure. ECG system 700 may be used at a hospital or may be a portable device for use wherever the patient may be. In another example, ECG system 700 may be an implantable biomedical device with leads implanted in various locations around the body of a patient. ECG system 700 may be part of or may be coupled with other implantable biomedical devices such as a cardiac pacemaker, an implantable cardioverter-defibrillator (ICD) or a cardiac resynchronization therapy (CRT) device.

ECG system 700 includes leads 702 and a main unit 704. Leads 702 may comprise any number and type of electrical lead. For example, leads 702 may comprise ten leads to be used with a standard 12-lead ECG. Leads 702 may be similar to leads 104a-j as illustrated in FIG. 1 and described previously. In another example, leads 702 may comprise implanted electrical leads, such as insulated wires placed throughout the body.

Main unit 704 may include an input module 706, a processor 708, a memory module 710 and a display 712. Input module 706 includes suitable circuitry and hardware to receive the signals from leads 702. As such, input module 706 may include components such as, for example, analog-to-digital converters, de-serializers, filters, and amplifiers. These various components may be implemented to condition the received signals to a more suitable form for further signal processing to be performed by processor 708.

It should be understood that in the case of the embodiment where ECG system 700 is an implantable biomedical device, display 712 may be replaced with a transceiver module configured to send and receive signals such as radio frequency (RF), optical, inductively coupled, or magnetic signals. In one example, these signals may be received by an external display for providing visual data related to measurements performed by ECG system 700 and analysis performed after receiving the signal and quantifying a spatio-temporal heterogeneity of the ECG signals based on the analysis.

Processor 708 may include one or more hardware microprocessor units. In an embodiment, processor 708 is configured to perform signal processing procedures on the signals received via input module 706. For example, processor 708 may perform the ECG heterogeneity procedures, such as R-wave and T-wave heterogeneity analysis for prediction of efficacy of implanting a pacemaker or CRT device in a patient. Processor 708 may also comprise a field-programmable gate array (FPGA) that includes configurable logic. The configurable logic may be programmed to perform the ECG heterogeneity procedures using configuration code stored in memory module 710. Likewise, processor 708 may be programmed via instructions stored in memory module 710.

Memory module 710 may include any type of memory including random access memory (RAM), read-only memory (ROM), electrically-erasable programmable read-only memory (EEPROM), FLASH memory, etc. Furthermore, memory module 710 may include both volatile and non-volatile memory. For example, memory module 710 may contain a set of coded instructions in non-volatile memory for programming processor 708. The calculated baseline signal may also be stored in either the volatile or non-volatile memory depending on how long it is intended to be maintained. Memory module 710 may also be used to save data related to the calculated TWH or RWH, including trend data for each.

In an embodiment, main unit 704 includes display 712 for providing a visual representation of the received signals from leads 702. Display 712 may utilize any of a number of different display technologies such as, for example, liquid crystal display (LCD), light emitting diode (LED), plasma or cathode ray tube (CRT). An ECG signal from each of leads 702 may be displayed simultaneously on display 712. In another example, a user may select which ECG signals to display via a user interface associated with main unit 704. Display 712 may also be used to show data trends over time, such as displaying trends of the calculated RWH and TWH.

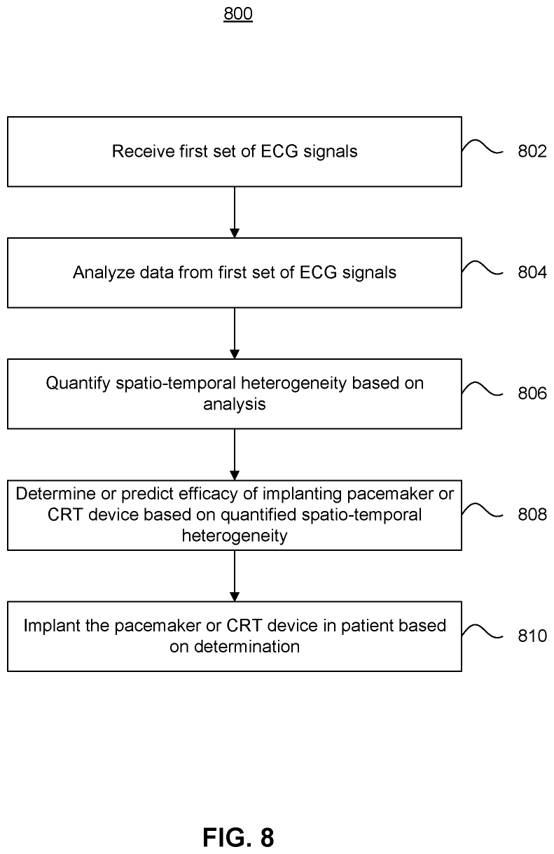

FIG. 8 illustrates a flowchart depicting a method 800 for predicting efficacy of implanting a pacemaker or CRT device in a patient, according to embodiments of the present disclosure. Method 800 may be performed by the various components of ECG system 700. It is to be appreciated that method 800 may not include all operations shown or perform the operations in the order shown.

Method 800 begins at step 802 where a first set of ECG signals of a patient is received. In particular, the ECG signals may be monitored via leads such as those illustrated in FIG. 1, or via implantable leads, and received by an ECG recording device or ECG system, such as ECG system 700. In some cases, the first set of ECG signals may be obtained via spatially separated leads such as V1, V2, and V3 or V4, V5, and V6 of a standard 12-lead ECG.

At step 802, the data from the first set of ECG signals may be analyzed. The analysis may be implemented by the ECG system 700 and may include second central moment analysis techniques.

At step 804, the spatio-temporal heterogeneity of the first set of ECG signals may be quantified based on the analysis. In particular, the processor 708 of the ECG system 700 may calculate at least one of the R-wave heterogeneity (RWH) and T-wave heterogeneity (TWH) of the first set of ECG signals.

At step 806, the efficacy of implanting a pacemaker or CRT device in the patient may be determined based on the quantified spatio-temporal heterogeneity. In particular, if the patient has a lower range of TWH that is below a predetermined threshold or range of threshold values, then the EEG system 700 may generate a prediction that the patient is more likely to benefit from CRT implantation than other patients.

At step 808, a pacemaker or CRT device may be implanted in the patient based on the determination. In some embodiments, the quantified spatio-temporal heterogeneity, including parameters and lead sites identified during quantification of the ECG heterogeneity data, may be utilized to guide placement of the device in the patient.

### Exemplary Computer Implementation

FIGS. 1-8 as described herein are illustrative examples allowing an explanation of the present invention. It should be understood that embodiments of the present invention could be implemented in hardware, firmware, software, or a combination thereof. In such an embodiment, the various components and steps would be implemented in hardware, firmware, and/or software to perform the functions of the present invention. That is, the same piece of hardware, firmware, or module of software could perform one or more of the illustrated blocks (i.e., components or steps).

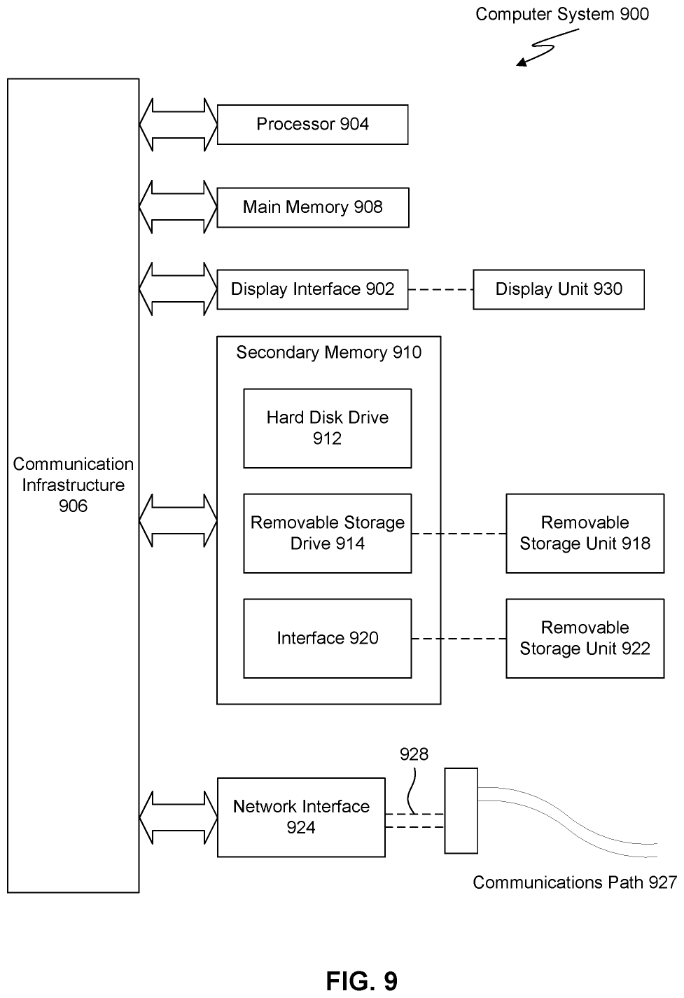

The present invention may be implemented in one or more computer systems capable of carrying out the functionality described herein. Referring to FIG. 9, an example computer system 900 useful in implementing the present invention is shown. Various embodiments of the invention are described in terms of this example computer system 900. After reading this description, it will become apparent to one skilled in the relevant art(s) how to implement the invention using other computer systems and/or computer architectures.

The computer system 900 includes one or more processors, such as processor 904. The processor 904 is connected to a communication infrastructure 906 (e.g., a communications bus, crossover bar, or network).

Computer system 900 may include a display interface 902 that forwards graphics, text, and other data from the communication infrastructure 906 (or from a frame buffer not shown) for display on the display unit 930.

Computer system 900 also includes a main memory 908, preferably random access memory (RAM), and may also include a secondary memory 910. The secondary memory 910 may include, for example, a hard disk drive 912 and/or a removable storage drive 914, representing a floppy disk drive, a magnetic tape drive, an optical disk drive, etc. The removable storage drive 914 reads from and/or writes to a removable storage unit 918 in a well-known manner. Removable storage unit 918, represents a floppy disk, magnetic tape, optical disk, etc. which is read by and written to removable storage drive 914. As will be appreciated, the removable storage unit 918 includes a computer usable storage medium having stored therein computer software (e.g., programs or other instructions) and/or data.

In alternative embodiments, secondary memory 910 may include other similar means for allowing computer software and/or data to be loaded into computer system 900. Such means may include, for example, a removable storage unit 922 and an interface 920. Examples of such may include a program cartridge and cartridge interface (such as that found in video game devices), a removable memory chip (such as an EPROM, or PROM) and associated socket, and other removable storage units 922 and interfaces 920 which allow software and data to be transferred from the removable storage unit 922 to computer system 900.

Computer system 900 may also include a communications interface 924. Communications interface 924 allows software and data to be transferred between computer system 900 and external devices. Examples of communications interface 924 may include a modem, a network interface (such as an Ethernet card), a communications port, a PCMCIA slot and card, etc. Software and data transferred via communications interface 924 are in the form of signals 928 which may be electronic, electromagnetic, optical, or other signals capable of being received by communications interface 924. These signals 928 are provided to communications interface 924 via a communications path (i.e., channel) 926. Communications path 926 carries signals 928 and may be implemented using wire or cable, fiber optics, a phone line, a cellular phone link, an RF link, free-space optics, and/or other communications channels.

In this document, the terms “computer program medium” and “computer usable medium” are used to generally refer to media such as removable storage unit 918, removable storage unit 922, a hard disk installed in hard disk drive 912, and signals 928. These computer program products are means for providing software to computer system 900. The invention is directed to such computer program products.

Computer programs (also called computer control logic or computer readable program code) are stored in main memory 908 and/or secondary memory 910. Computer programs may also be received via communications interface 924. Such computer programs, when executed, enable the computer system 900 to implement the present invention as discussed herein. In particular, the computer programs, when executed, enable the processor 904 to implement the processes of the present invention described above. Accordingly, such computer programs represent controllers of the computer system 900.

In an embodiment where the invention is implemented using software, the software may be stored in a computer program product and loaded into computer system 900 using removable storage drive 914, hard disk drive 912, interface 920, or communications interface 924. The control logic (software), when executed by the processor 904, causes the processor 904 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to one skilled in the relevant art(s).

In yet another embodiment, the invention is implemented using a combination of both hardware and software.

In one example embodiment, the present invention may be implemented in a computer-based monitor unit for use in a clinical setting. In another embodiment, the present invention may be implemented in an ambulatory unit akin to a Holter monitor, personal computing device, or similar portable device. In yet another embodiment, the present invention may be implemented in an implantable medical device such as an implantable cardioverter defibrillator (ICD).

The foregoing description of the specific embodiments will so fully reveal the general nature of the invention that others can, by applying knowledge within the skill of the art (including the contents of the documents cited and incorporated by reference herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the art.

While various embodiments of the present invention have been described above, it should be understood that they have been presented by way of example, and not limitation. It will be apparent to one skilled in the relevant art(s) that various changes in form and detail can be made therein without departing from the spirit and scope of the invention. Thus, the present invention should not be limited by any of the above-described exemplary embodiments, but should be defined only in accordance with the following claims and their equivalents.

What is claimed is:

1. A method of determining or predicting efficacy of implanting a pacemaker or cardiac resynchronization therapy (CRT) device in a patient, the method comprising:
receiving a first set of electrocardiogram (ECG) signals associated with a heart of the patient from spatially separated leads;
analyzing data from the first set of ECG signals;
quantifying a spatio-temporal heterogeneity of the first set of ECG signals based on the analysis; and
determining or predicting efficacy of implanting the pacemaker or CRT device in the patient based on the quantified spatio-temporal heterogeneity.

2. The method of claim 1, further comprising:
implanting the pacemaker or CRT device in the patient based on the determination or prediction.

3. The method of claim 2, further comprising:
using the spatio-temporal heterogeneity to guide placement of the pacemaker or CRT device in the patient.

4. The method of claim 1, wherein quantifying the spatio-temporal heterogeneity of the first set of ECG signals comprises:
calculating the R-wave heterogeneity (RWH) and T-wave heterogeneity (TWH) of the first set of ECG signals.

5. The method of claim 1, wherein the spatially separated leads include leads V1, V2, and V3 of a standard 12-lead ECG.

6. The method of claim 1, wherein the spatially separated leads include leads V4, V5, and V6 of a standard 12-lead ECG.

7. An electrocardiogram (ECG) system for determining or predicting efficacy of implanting a pacemaker or cardiac resynchronization therapy (CRT) device in a patient, the system comprising:
an input module configured to receive ECG signals from spatially separated leads; and
a processor configured to:
receive a first set of ECG signals associated with a heart of the patient from spatially separated leads;
analyze data from the first set of ECG signals;
quantify a spatio-temporal heterogeneity of the first set of ECG signals based on the analysis; and
determine or predict efficacy of implanting the pacemaker or CRT device in the patient based on the quantified spatio-temporal heterogeneity.

8. The system of claim 7, wherein the pacemaker or CRT device is implanted in the patient based on the determination or prediction.

9. The system of claim 8, wherein the processor is further configured to use the spatio-temporal heterogeneity to guide placement of the pacemaker or CRT device in the patient.

10. The system of claim 7, wherein the processor is further configured to quantify the spatio-temporal heterogeneity of the first set of ECG signals by calculating the R-wave heterogeneity (RWH) and T-wave heterogeneity (TWH) of the first set of ECG signals.

11. The system of claim 7, wherein the spatially separated leads include leads V1, V2, and V3 of a standard 12-lead ECG.

12. The system of claim 7, wherein the spatially separated leads include leads V4, V5, and V6 of a standard 12-lead ECG.

13. A computer program product stored on a computer readable media, including a set of instructions that, when executed by a computing device, perform a method of determining or predicting efficacy of implanting a pacemaker or cardiac resynchronization therapy (CRT) device in a patient, comprising:
receiving a first set of electrocardiogram (ECG) signals associated with a heart of the patient from spatially separated leads;
analyzing data from the first set of ECG signals;
quantifying a spatio-temporal heterogeneity of the first set of ECG signals based on the analysis; and
determining or predicting efficacy of implanting the pacemaker or CRT device in the patient based on the quantified spatio-temporal heterogeneity.

14. The computer program product of claim 13, wherein the method further comprises:
implanting the pacemaker or CRT device in the patient based on the determination or prediction.

15. The computer program product of claim 14, wherein the method further comprises:
using the spatio-temporal heterogeneity to guide placement of the pacemaker or CRT device in the patient.

16. The computer program product of claim 13, wherein the quantifying the spatio-temporal heterogeneity of the first set of ECG signals comprises:
calculating the R-wave heterogeneity (RWH) and T-wave heterogeneity (TWH) of the first set of ECG signals.

17. The computer program product of claim 13, wherein the spatially separated leads include leads V1, V2, and V3 of a standard 12-lead ECG.

18. The computer program product of claim 13, wherein the spatially separated leads include leads V4, V5, and V6 of a standard 12-lead ECG.
